(19) United States
(12) Patent Application Publication
(10) Pub. No.: US 2003/0236243 A1
(43) Pub. Date: Dec. 25, 2003

(54) Sterically-awkward beta-lactamase inhibitors

(76) Inventors: Brian K. Shoichet, San Francisco, CA (US); Larry C. Blaszczak, Indianapolis, IL (US)

Correspondence Address: REINHART BOERNER VAN DEUREN S.C., ATTN: LINDA GABRIEL, DOCKET COORDINATOR, 1000 NORTH WATER STREET, SUITE 2100, MILWAUKEE, WI, 53202, US

(21) Appl. No.: 10/438,280

(22) Filed: May 14, 2003

Related U.S. Application Data
(60) Provisional application No. 60/380,411, filed on May 14, 2002.

(51) Int. Cl. A61K 31/545; A61K 31/43; C07D 501/14; C07D 499/80

(52) U.S. Cl. 514/196; 514/202; 540/222; 540/325

(57) ABSTRACT

6(7)-β-substituted β-lactam compounds as inhibitors of β-lactamase activity.

This application claims priority benefit from provisional application serial No. 60/380,411 filed on May 14, 2002, the entirety of which is incorporated herein by reference.

The United States Government has certain rights to this invention pursuant to Grant No. GM63815 from the National Institutes of Health to Northwestern University.

### BACKGROUND OF THE INVENTION

The impact of bacterial resistance on antimicrobial chemotherapy is a well documented public health problem. Among the classes of antibiotic compositions hardest hit are β-lactams, such as the penicillins and cephalosporins, which are also among the most prescribed. The most widespread resistance mechanism against these antibiotics is the expression of β-lactamases, which hydrolyze such compositions, inactivating them. All β-lactams share the same core four-membered lactam ring from which they take their name. It is this core structure that is recognized and hydrolyzed by β-lactamases. Nevertheless, molecular substitutions distant from the lactam ring can convert a β-lactamase substrate into a β-lactamase inhibitor. For instance, whereas penicillin G and cephalothin are excellent substrates for class C β-lactamases such as AmpC, cloxacillin and ceftazidime are either inhibitors of or very poor substrates for these enzymes.

Structural studies of the prior art suggest that some β-lactams act as inhibitors because they are sterically “awkward” and cannot be fit into a catalytically competent conformation in the active site of class C β-lactamases. Thus, although they rapidly form covalent adducts in the initial acylation phase of the hydrolytic reaction, bulky substituents on the R₁ side chains of these β-lactams force them to adopt catalytically incompetent configurations within the acyl adduct, preventing the deacylation step of the reaction from taking place, effectively trapping the enzyme. The mechanism of these inhibitors may thus be distinguished from mechanism-based “suicide” substrates, such as clavulanate, which rely on secondary chemical reactions within the enzyme, and from classical non-covalent substrate-based inhibitors, which rely on steric complementarity to the enzyme site. Inhibitors that can acylate β-lactamases, but which cannot easily adopt the catalytically-competent configuration necessary for deacylation, can be referred to as “awkward inhibitors”.

An example of a chemical group/substituent that appears to force an awkward configuration in the binding site of AmpC is the 2-amino-4-thiazolyl methoxyimino (ATMO) group common to the 3^{rd} generation cephalosporins, such as cefotaxime and ceftazidime. The structure of the acyl-adduct of ceftazidime with AmpC initially suggested that this ATMO group, which occurs at the distal end of the molecule, forces the dihydrothiazine ring of the cephalosporin into a configuration where it destabilizes the formation of the deacylation transition state, thereby making it an inhibitor or very poor substrate of AmpC. However, in counterpoint to these preliminary structural studies, a series of elegant enzymological studies of 3^{rd} generation cephalosporins has suggested that at least part of the ability of such compositions to inhibit class C β-lactamases is conferred by an internal electronic rearrangement that displaces the R₂ side chain peculiar to cephalosporin structures.

R₂ groups at the C3 position of the dihydrothiazine ring of cephalosporins have become the subject of much synthetic effort in novel β-lactam design (FIG. 1A). When the lactam ring is opened as a result of attack by the serine nucleophile (Ser64 in AmpC), the lone pair electrons on the formerly lactam nitrogen are free to rearrange, leading to the departure of the displaceable R₂ side chain (FIG. 2). The resulting ring structure is thought to be more stable to hydrolytic attack. In cephalosporins that are rapidly hydrolyzed, this rearrangement happens slower than hydrolysis of the acyl-enzyme species and consequent product formation, and thus does not affect how well the β-lactam might inhibit the enzyme. For molecules that are intrinsically slow to deacylate, for instance because of steric interactions in their R₁ side chains, this rearrangement will happen before deacylation, thereby further stabilizing the acyl adduct against deacylation. In such cases, the β-lactam will be a slow substrate, to the point where it may be considered an inhibitor of the β-lactamase.

### BRIEF DESCRIPTION OF THE FIGURES

FIG. 1A. AmpC β-lactamase inhibitors of the prior art, exhibiting displaceable R₂ groups.

FIG. 1B. Representative R₁ side chains/substituents shown in conjunction with penicillin and carbacephem core structures, in accordance with various compositional aspects of this invention.

FIG. 2. Without limitation, but with reference to the prior art, two possible routes of cephalosporin hydrolysis by class C β-lactamases. The center scheme shows an overall reaction pathway, involving nucleophilic attack by a catalytic serine residue and eventual deacylation leading to hydrolyzed antibiotic, departed R₂ leaving group, and free enzyme. The scheme on the left depicts a case where deacylation occurs rapidly, freeing the hydrolyzed antibiotic, which subsequently undergoes the electronic rearrangement that leads to the departure of the R₂ group. The scheme on the right depicts a case where deacylation occurs more slowly and so the electronic rearrangement and departure of the R₂ group occurs while the β-lactam is still covalently bound to the enzyme.

FIGS. 3A-B. 2|F_{O}|-|F_{C}| electron density of the refined models of AmpC in complex with (A) amoxicillin and (B) ATMO-penicillin, contoured at 1σ. The deacylating water, Wat402, is shown as a sphere. This figure was generated using SETOR. J. Mol. Graph. 1993; 11(2):134-8, 127-8.

FIGS. 4A-B. The active site of AmpC covalently bound to (A) amoxicillin and (B) ATMO-penicillin. Dashed lines represent hydrogen bond interactions.—Wat402 is the deacylating water.

FIGS. 5A-C. (A) AmpC in complex with the substrates amoxicillin, cephalothin and loracarbef. AmpC complexes with (B) amoxicillin and (C) ATMO-penicillin overlaid with the deacylation transition state analog ceftazidime boronic acid. (The dimethyl-carboxylate group of the boronic acid has been eliminated for clarity.) The distance between the ring nitrogen and the presumed position of the hydrolytic water in the deacylation high-energy intermediate is indicated.

### SUMMARY OF THE INVENTION

As mentioned above, β-lactamase enzymes are important therapeutic targets in a broad range of bacterial species because of their prominent role in resistance to the β-lactam class of antibiotics, including the penicillins and cephalosporins. The search for new β-lactamase inhibitors has followed several strategies. The present invention relates to the successful design and synthesis of new β-lactams as β-lactamase inhibitors and also as antibiotics against clinically relevant β-lactamase-expressing pathogens. Certain embodiments of the compositions/inhibitors of this invention can exemplify the concept of “awkward” inhibitors—compounds that are believed able to bind covalently to the enzyme but subsequently hold or trap the enzyme in this covalent linkage. Through kinetic analysis, it has been shown the presence of large, bulky substituents that clash with conserved active site residues is at least in part responsible for this trapping. Atomic resolution X-ray crystal structures also show that this steric clash can force the inhibitor to adopt a catalytically incompetent conformation which blocks the final steps in catalysis that would normally free the enzyme from the hydrolyzed reaction product.

Accordingly, it is an object of this invention to provide for the use and/or transferability of sterically-demanding, bulky substituents among different families of β-lactams for converting substrates of β-lactamases into inhibitors at nanomolar concentration levels. More particularly, it is an object of this invention to provide for substitution of a bulky R₁ side chain in the 6(7)-β position sufficient to convert substrate β-lactams into potent inhibitors of class C β-lactamases. Broadly, this objective and various other aspects of this invention can be applied to the design of inhibitors for other enzymes that operate via a mechanism involving a covalent intermediate. Awkward inhibitors whose core structures resemble their target enzyme's normal substrates can form an acyl adduct with the enzyme, then block subsequent steps in the catalytic mechanism, effectively trapping the enzyme in its covalently-bound state. Steric “awkwardness”, as illustrated herein, provides a rationale for the design of new β-lactamase inhibitors. Accordingly, it is also an object of this invention to provide compounds for use as anti-resistance antibiotics, such as -against pathogenic, β-lactamase producing bacteria including E. cloacae, E. coli, and S. aureus, all of which are resistant to most other β-lactam antibiotics.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention.

Other objects, features, benefits and advantages of the present invention will be apparent from this summary and its descriptions of certain embodiments, and will be readily apparent to those skilled in the art having knowledge of various lactamase inhibitors, procedures for their design and production, and antibiotic treatments and related methods. Such objects, features, benefits and advantages will be apparent from the above as taken into conjunction with the accompanying examples, data, figures and all reasonable inferences to be drawn therefrom.

In consideration of the above and in conjunction with the examples and data following hereafter, the present invention includes, in part, compounds/compositions providing a β-lactamase inhibitory effect. Such compounds/compositions comprise a β-lactam molecular core structure, such a structure including but not limited to a penicillin (e.g., amoxicillin), a carbacephem (e.g., loracarbef), an oxacephem (e.g., moxalactam), or a carbapenem (e.g., imipenem) core structure, such a structure substituted at the 6(7)-β position, at the lactam position adjacent to the acyl carbon, with a side chain/group/substituent and moiety imparting steric bulk and/or effect to such a compound/composition in the context of its complex with a β-lactamase enzyme. In certain preferred embodiments, such a group or substituent includes an aminothiazole oxime component in conjunction with an alkoxyimino moiety. As illustrated elsewhere herein, a methoxyimino moiety can be utilized with good effect as can other moieties structurally-varied depending on length, branching and/or substitution of the corresponding alkyl component thereof. Such moieties can take into consideration a range of steric and/or charge factors. Accordingly, a dimethylcarboxylate analog, as illustrated herein, has also been shown to be efficacious. In certain other embodiments, an N-substituted benzylamine group or substituent can be used and/or incorporated into the structure of an inhibitor compound, as provided elsewhere herein.

For purposes of the present compounds, compositions and/or methods, the following expression(s) and word(s), unless otherwise indicated, will be understood as having the meanings ascribed thereto by those skilled in the art or as otherwise indicated with respect thereto:

“β-lactam core structure” or “core structure” means a structure containing a 5- or 6-member fused ring β-lactam structure, substituted at the 6(7)-β position. Representative structures, without necessary regard to stereochemistry, charge or degree of protonation, include but are not limited to

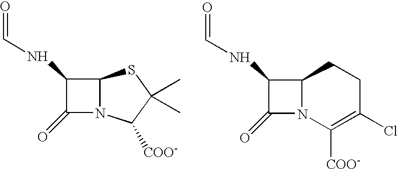

“N-substituted benzylamine” means a substituent structure of a β-lactam core structure coupled or bonded thereto at the benzyl carbon, N-substituted and optionally phenyl-substituted. Representative N-substituted benzylamine structures include but are not limited to

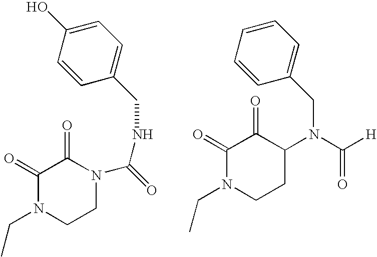

such substituent structures as can be available through corresponding N-alkylation or acylation or other such modification of an amoxicillin or loracarbef core structure.

“Aminothiazole oxime” means a substituent of a β-lactam core structure coupled or bonded thereto at the iminocarbon and O-substituted. Representative aminothiazole oxime structures include but are not limited to

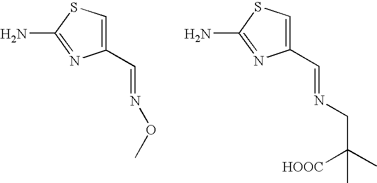

such substituent structures as can be utilized via corresponding modification of a penicillin or a carbacephem core structure.

While certain compounds/compositions of this invention are described in conjunction with several representative side chains/groups/substituents, together with their various structural components and/or moieties, it will be understood by those skilled in the art made aware of this invention that such compounds/compositions can comprise other analogous substituted β-lactams. Core lactam structures can include those mentioned above. Suitable substitutions, whether an AMTO substituent, an analogous aminothiazole oxime substituent, or another substituent providing desired functional or steric effect, can be determined in a straight-forward manner by those skilled in the art without undue experimentation following the examples and criteria provided herein. A suitable side chain/group/substituent, available as provided herein and/or through synthetic techniques known in the art, can impart a steric impact on the β-lactam core, in the context of lactamase complexation, sufficient to impose a confirmation upon the structure whereby further enzymatic activity is inhibited.

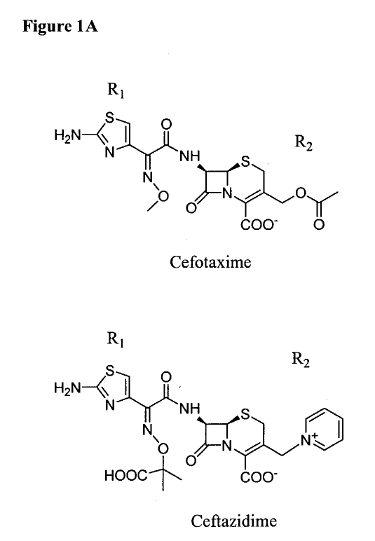

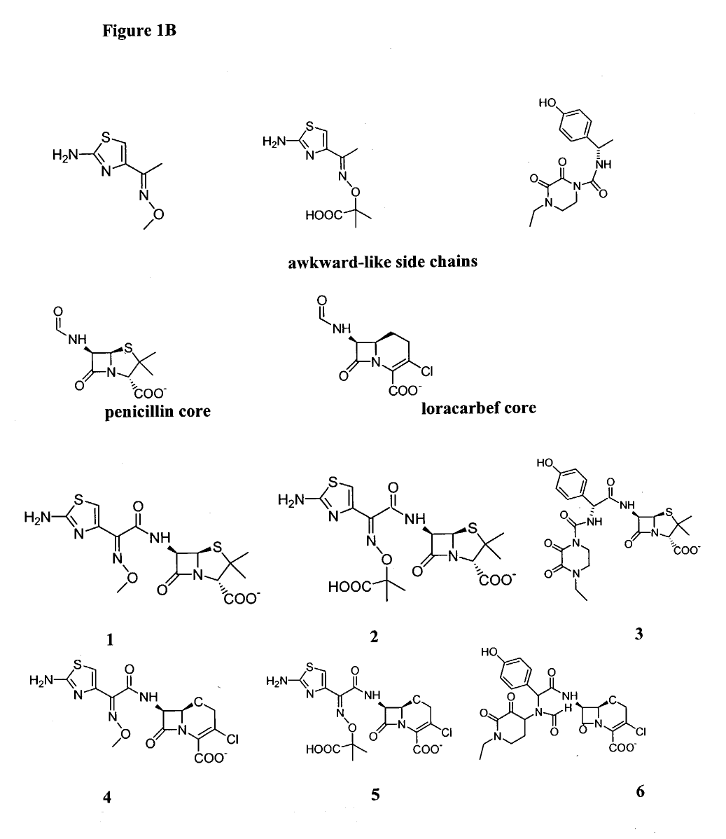

Compounds 1-6 of this invention are provided in FIG. 1B, illustrated with their component core and substituent formulae and/or structures. With regard to certain embodiments, the present invention provides results which are both surprising and unexpected. Contrary to previous findings and literature publications, the presence of an R₁ side chain/group/substituent, without a corresponding R₂ structure, is sufficient to convert a β-lactam composition from what would nominally be a β-lactamase substrate into a potent inhibitor. For example, whereas penicillin is a β-lactamase substrate, R₁-substituted penicillin inhibits Class C β-lactamases, while appearing to avoid Class A β-lactamases. Such a structural modification can be used to activate a β-lactam compound against β-lactamase expressing bacteria.

Accordingly, the present invention can also include a method of using β-lactam substitutions, described above, to treat and/or inhibit β-lactamase expressing bacteria. Such a method includes (1) providing a compound/composition in accordance with this invention, having a β-lactam molecular core structure substituted as further described herein; and (2) treating and/or contacting a β-lactamase or such an expressing bacteria with such compound/composition. Bacteria producing such a β-lactamase, as can be treated with the substituted β-lactams of this invention, include those caused by both gram-positive and gram-negative bacteria, for example, bacteria of the genus Staphylococcus (such as Staphylococcus aureus and Staphylococcus epidermis), Streptococcus (such as Streptococcus agalactine, Streptococcus pneumoniae and Streptococcus faecalis), Micrococcus (such as Micrococcus luteus), Bacillus (such as Bacillus subtilis), Listerella (such as Listerella monocytogenes), Escherichia (such as Escherichia coli), Klebsiella (such as Klebsiella pneumoniae), Proteus (such as Proteus mirabilis and Proteus vulgaris), Salmonella (such as Salmonella typhosa), Shigella (such as Shigella sonnei), Enterobacter (such as Enterobacter aerogenes and Enterobacter facium), Serratia (such as Serratia marcescens), Pseudomonas (such as Pseudomonas aeruginosa), Acinetobacter (such as Acinetobacter anitratus), Nocardia (such as Nocardia autotrophica), and Mycobacterium (such as Mycobacterium fortuitum).

Related methods and/or treatments, in accordance with this invention, are preferably effected in conjunction with but without limitation to compounds 1-6 illustrated in FIG. 1B. More generally, as can be incorporated into a treatment protocol or methodology, this invention can comprise inhibitor compounds of the formula

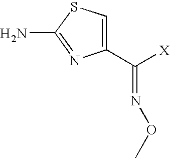

wherein R₂ is a moiety selected from alkyl and substituted alkyl moieties and X comprises a β-lactam core structure heretofore neither disclosed nor suggested by the prior art. More specifically, such a core structure is not a cephalosporin and is not a monobactam. Consistent with the preceding, X can comprise a penicillin core structure; regardless, R₂ can be alkyl. In certain other embodiments, X can comprise a carbacephem core structure, and R₂ can be a carboxy-substituted alkyl. As illustrated by such penicillin and carbacephem embodiments, this invention can utilize with advantageous effect β-lactam core structures without or absent a displaceable substituent group or moiety at the C-3 position thereof—in contrast to and as distinguished from the prior art. In various other embodiments, such compounds or pharmaceutically-accepted salts thereof can also be used in combination with other β-lactam antibiotics, including but not limited to third generation cephalosporins, such as cefotaxime, ceftazidine, and ceftriaxone.

With respect to either the compounds, compositions and/or methods of the present invention, the aforementioned moieties or components can comprise, consist of, or consist essentially of any of the aforementioned substituents and functional groups thereof. Each such compound or moiety/substituent thereof is distinguishable, characteristically contrasted, and can be practiced in conjunction with the present invention separate and apart from another. Accordingly, it should be understood that the inventive compounds, compositions and/or methods, as illustratively disclosed herein, can be practiced or utilized in the absence of any one compound, moiety and/or substituent which may or may not be disclosed, referenced or inferred herein, the absence of which may or may not be specifically disclosed, referenced or inferred herein.

The compounds of this invention may contain an acidic or basic functional group and are, thus, capable of forming pharmaceutically-acceptable salts with pharmaceutically-acceptable acids and bases. The term “pharmaceutically-acceptable salts” refers to the relatively non-toxic, inorganic and organic acid and base addition salts of such compounds. These salts can be prepared by reacting the purified compound with a suitable acid or base. Suitable bases include the hydroxide, carbonate or bicarbonate of a pharmaceutically-acceptable metal cation, ammonia, or a pharmaceutically-acceptable organic primary, secondary or tertiary amine. Representative alkali or alkaline earth salts include the lithium, sodium, potassium, calcium, magnesium, and aluminum salts and the like. Representative organic amines useful for the formation of base addition salts include ethylamine, diethylamine, ethylenediamine, ethanolamine, diethanolamine, piperazine and the like. Representative acid addition salts include the hydrobromide, hydrochloride, sulfate, phosphate, nitrate, acetate, valerate, oleate, palmitate, stearate, laurate, benzoate, lactate, phosphate, tosylate, citrate, maleate, fumarate, succinate, tartrate, napthalate, mesylate, glucoheptonate, lactobionate, and laurylsulphonate salts and the like.

As mentioned above, the compounds of this invention, and the pharmaceutically-acceptable salts thereof, are inhibitors of β-lactamases. Assays for the inhibition of β-lactamase activity are well known in the art. For instance, the ability of a compound to inhibit β-lactamase activity in a standard enzyme inhibition assay may be used (see, e.g., Example 1 below and M. G. Page, Biochem J. 295 (Pt. 1) 295-304 (1993)). β-lactamases for use in such assays may be purified from bacterial sources or, preferably, are produced by recombinant DNA techniques, since genes and cDNA clones coding for many β-lactamases are known. See, e.g., S. J. Cartwright and S. G. Waley, Biochem J. 221, 505-512 (1984). Alternatively, the sensitivity of bacteria known, or engineered, to produce a β-lactamase to an inhibitor may be determined. Other bacterial inhibition assays include agar disk diffusion and agar dilution. See, e.g., W. H. Traub & B. Leonhard, Chemotherapy 43, 159-167 (1997). Thus, a β-lactamase can be inhibited by contacting the β-lactamase enzyme with an effective amount of an inventive compound or by contacting bacteria that produce the β-lactamase enzymes with an effective amount of such a compound so that the β-lactamase in the bacteria is contacted with the inhibitor. The contacting may take place in vitro or in vivo. “Contacting” means that the β-lactamase and the inhibitor are brought together so that the inhibitor can bind to the β-lactamase. Amounts of a compound effective to inhibit a β-lactamase may be determined empirically, and making such determinations is within the skill in the art. Inhibition includes both reduction and elimination of β-lactamase activity.

The present compounds, and the pharmaceutically-acceptable salts thereof, can be used to treat β-lactam-antibiotic-resistant bacterial infections. “β-lactam-antibiotic-resistant bacterial infection” is used herein to refer to an infection caused by bacteria resistant to treatment with one or more β-lactam antibiotics due primarily to the action of a β-lactamase. Resistance to β-lactam antibiotics can be determined by standard antibiotic sensitivity testing. The presence of β-lactamase activity can be determined as is well known in the art (see above). Alternatively, the sensitivity of a particular bacterium to the combination of an inventive compound, or a pharmaceutically-acceptable salt thereof, and a β-lactam antibiotic can be determined by standard antibiotic sensitivity testing methods.

To treat a β-lactam resistant bacterial infection, an animal or subject suffering from such an infection is given an effective amount of a compound of this invention, or a pharmaceutically-acceptable salt thereof, and an effective amount of a β-lactam antibiotic. Such a compound, or a pharmaceutically-acceptable salt thereof, and the β-lactam antibiotic may be given at different times or given together. When administered together, they may be contained in separate pharmaceutical compositions or they may be in the same pharmaceutical composition.

Many suitable β-lactam antibiotics are known in the art, including but not limited to the cephalosporins, penicillins, monobactams, carbapenems, and carbacephems. β-lactam antibiotics are effective (in the absence of resistance) against a wide range of bacterial infections. These include those caused by both gram-positive and gram-negative bacteria, for example, bacteria of the genus Staphylococcus (such as Staphylococcus aureus and Staphylococcus epidermidis), Streptococcus (such as Streptococcus agalactine, Streptococcus penumoniae and Streptococcus faecalis), Micrococcus (such as Micrococcus luteus), Bacillus (such as Bacillus subtilis), Listerella (such as Listerella monocytogenes), Escherichia (such as Escherichia coli), Klebsiella (such as Klebsiella pneumoniae), Proteus (such as Proteus mirabilis and Proteus vulgaris), Salmonella (such as Salmonella typhosa), Shigella (such as Shigella sonnei), Enterobacter (such as Enterobacter aerogenes and Enterobacter cloacae), Serratia (such as Serratia marcescens), Pseudomonas (such as Pseudomonas aeruginosa), Acinetobacter such as Acinetobacter anitratus), Nocardia (such as Nocardia autotrophica), and Mycobacterium (such as Mycobacterium fortuitum). Effective doses and modes of administration of β-lactam antibiotics are known in the art or may be determined empirically or as described below for such compounds.

To treat an animal/subject suffering from a β-lactam-antibiotic-resistant bacterial infection, an effective amount of one or more of the present compounds, or a pharmaceutically-acceptable salt thereof, is administered in combination with a β-lactam antibiotic. Effective dosage forms, modes of administration and dosage amounts may be determined empirically, and making such determinations is within the skill of the art. It is understood by those skilled in the art that the dosage amount will vary with the activity of the particular compound employed, the severity of the bacterial infection, the route of administration, the rate of excretion of the compound, the duration of the treatment, the identity of any other drugs being administered to the animal/subject, the age, size and species of the animal, and like factors well known in the medical and veterinary arts. In general, a suitable daily dose will be that amount which is the lowest dose effective to produce a therapeutic effect. The total daily dosage will be determined by an attending physician or veterinarian within the scope of sound medical judgment. If desired, the effective daily dose of such a compound, or a pharmaceutically-acceptable salt thereof, maybe administered as two, three, four, five, six or more sub-doses, administered separately at appropriate intervals throughout the day. Treatment of a β-lactam-antibiotic-resistant bacterial infection according to the invention, includes mitigation, as well as elimination, of the infection. Animals treatable according to the invention include mammals. Mammals treatable according to the invention include dogs, cats, other domestic animals, and humans.

Compounds of this invention may be administered to an animal/patient for therapy by any suitable route of administration, including orally, nasally, rectally, intravaginally, parenterally, intracisternally and topically, as by powders, ointments or drops, including buccally and sublingually. The preferred routes of administration are orally and parenterally.

While it is possible for the active ingredient(s) (one or more compounds of this invention and/or pharmaceutically-acceptable salts thereof, alone or in combination with a β-lactam antibiotic) to be administered alone, it is preferable to administer the active ingredient(s) as a pharmaceutical formulation (composition). The pharmaceutical compositions of the invention comprise the active ingredient(s) in admixture with one or more pharmaceutically-acceptable carriers and, optionally, with one or more other compounds, drugs or other materials. Each carrier must be “acceptable” in the sense of being compatible with the other ingredients of the formulation and not injurious to the patient.

Pharmaceutical formulations of the present invention include those suitable for oral, nasal, topical (including buccal and sublingual), rectal, vaginal and/or parenteral administration. Regardless of the route of administration selected, the active ingredient(s) are formulated into pharmaceutically-acceptable dosage forms by conventional methods known to those of skill in the art.

The amount of the active ingredient(s) which will be combined with a carrier material to produce a single dosage form will vary depending upon the host being treated, the particular mode of administration and all of the other factors described above. The amount of the active ingredient(s) which will be combined with a carrier material to produce a single dosage form will generally be that amount of the active ingredient(s) which is the lowest dose effective to produce a therapeutic effect.

Methods of preparing pharmaceutical formulations or compositions include the step of bringing the active ingredient(s) into association with the carrier and, optionally, one or more accessory ingredients. In general, the formulations are prepared by uniformly and intimately bringing the active ingredient(s) into association with liquid carriers, or finely divided solid carriers, or both, and then, if necessary, shaping the product.

Formulations of the invention suitable for oral administration may be in the form of capsules, cachets, pills, tablets, lozenges (using a flavored basis, usually sucrose and acacia or tragacanth), powders, granules, or as a solution or a suspension in an aqueous or nonaqueous liquid, or as an oil-in-water or water-in-oil liquid emulsion, or as an elixir or syrup, or as pastilles (using an inert base, such as gelatin and glycerin, or sucrose and acacia) and/or as mouth washes and the like, each containing a predetermined amount of the active ingredient(s). The active ingredient(s) may also be administered as a bolus, electuary or paste.

In solid dosage forms of the invention for oral administration (capsules, tablets, pills, dragees, powders, granules and the like), the active ingredient(s) is/are mixed with one or more pharmaceutically-acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethyl-cellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such as talc, calcium stearate, magnesium stearate, solid polyethylene glycols, sodium lauryl sulfate, and mixtures thereof; and (10) coloring agents. In the case of capsules, tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered active ingredient(s) moistened with an inert liquid diluent.

The tablets, and other solid dosage forms of the pharmaceutical compositions of the present invention, such as dragees, capsules, pills and granules, may optionally be scored or prepared with coatings and shells, such as enteric coatings and other coatings well known in the pharmaceutical-formulating art. They may also be formulated so as to provide slow or controlled release of the active ingredient(s) therein using, for example, hydroxypropylmethyl cellulose in varying proportions to provide the desired release profile, other polymer matrices, liposomes and/or microspheres. They may be sterilized by, for example, filtration through a bacteria-retaining filter. These compositions may also optionally contain opacifying agents and may be of a composition that they release the active ingredient(s) only, or preferentially, in a certain portion of the gastrointestinal tract, optionally, in a delayed manner. Examples of embedding compositions which can be used include polymeric substances and waxes. The active ingredient(s) can also be in microencapsulated form.

Liquid dosage forms for oral administration of the active ingredient(s) include pharmaceutically-acceptable emulsions, microemulsions, solutions, suspensions, syrups and elixirs. In addition to the active ingredient(s), the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, coloring, perfuming and preservative agents. Suspensions, in addition to the active ingredient(s), may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations of the pharmaceutical compositions of the invention for rectal or vaginal administration may be presented as a suppository, which may be prepared by mixing the active ingredient(s) with one or more suitable nonirritating excipients or carriers comprising, for example, cocoa butter, polyethylene glycol, a suppository wax or salicylate and which is solid at room temperature, but liquid at body temperature and, therefore, will melt in the rectum or vaginal cavity and release the active ingredient(s). Formulations of the present invention which are suitable for vaginal administration also include pessaries, tampons, creams, gels, pastes, foams or spray formulations containing such carriers as are known in the art to be appropriate.

Dosage forms for the topical or transdermal administration of the active ingredient(s) include powders, sprays, ointments, pastes, creams, lotions, gels, solutions, patches and inhalants. The active ingredient(s) may be mixed under sterile conditions with a pharmaceutically-acceptable carrier, and with any buffers, or propellants which may be required.

The ointments, pastes, creams and gels may contain, in addition to the active ingredient(s), excipients, such as animal and vegetable fats, oils, waxes, paraffins, starch, tragacanth, cellulose derivatives, polyethylene glycols, silicones, bentonites, silicic acid, talc and zinc oxide, or mixtures thereof. Powders and sprays can contain, in addition to the active ingredient(s), excipients such as lactose, talc, silicic acid, aluminum hydroxide, calcium silicates and polyamide powder, or mixtures of these substances. Sprays can additionally contain customary propellants such as chlorofluorohydrocarbons and volatile unsubstituted hydrocarbons, such as butane and propane.

Transdermal patches have the added advantage of providing controlled delivery of the active ingredient(s) to the body. Such dosage forms can be made by dissolving, dispersing or otherwise incorporating the active ingredient(s) in a proper medium, such as an elastomeric matrix material. Absorption enhancers can also be used to increase the flux of the active ingredient(s) across the skin. The rate of such flux can be controlled by either providing a rate-controlling membrane or dispersing the active ingredient(s) in a polymer matrix or gel.

Pharmaceutical compositions of this invention suitable for parenteral administration comprise the active ingredient(s) in combination with one or more pharmaceutically-acceptable sterile isotonic aqueous or nonaqueous solutions, dispersions, suspensions or emulsions, or sterile powders which may be reconstituted into sterile injectable solutions or dispersions just prior to use, which may contain antioxidants, buffers, solutes which render the formulation isotonic with the blood of the intended recipient or suspending or thickening agents.

Examples of suitable aqueous and nonaqueous carriers which may be employed in the pharmaceutical compositions of the invention include water, ethanol, polyols (such as glycerol, propylene glycol, polyethylene glycol, and the like), and suitable mixtures thereof, vegetable oils, such as olive oil, and injectable organic esters, such as ethyl oleate. Proper fluidity can be maintained, for example, by the use of coating materials, such as lecithin, by the maintenance of the required particle size in the case of dispersions, and by the use of surfactants.

These compositions may also contain adjuvants such as wetting agents, emulsifying agents and dispersing agents. It may also be desirable to include isotonic agents, such as sugars, sodium chloride, and the like in the compositions. In addition, prolonged absorption of the injectable pharmaceutical form may be brought about by the inclusion of agents which delay absorption such as aluminum monostearate and gelatin.

In some cases, in order to prolong the effect of the active ingredient(s), it is desirable to slow the absorption of the drug from subcutaneous or intramuscular injection. This may be accomplished by the use of a liquid suspension of crystalline or amorphous material having poor water solubility. The rate of absorption of the active ingredient(s) then depends upon its/their rate of dissolution which, in turn, may depend upon crystal size and crystalline form. Alternatively, delayed absorption of parenterally-administered active ingredient(s) is accomplished by dissolving or suspending the active ingredient(s) in an oil vehicle.

Injectable depot forms are made by forming microencapsule matrices of the active ingredient(s) in biodegradable polymers such as polylactide-polyglycolide. Depending on the ratio of the active ingredient(s) to polymer, and the nature of the particular polymer employed, the rate of release of the active ingredient(s) can be controlled. Examples of other biodegradable polymers include poly(orthoesters) and poly(anhydrides). Depot injectable formulations are also prepared by entrapping the active ingredient(s) in liposomes or microemulsions which are compatible with body tissue. The injectable materials can be sterilized for example, by filtration through a bacterial-retaining filter.

The formulations may be presented in unit-dose or multi-dose sealed containers, for example, ampoules and vials, and may be stored in a lyophilized condition requiring only the addition of the sterile liquid carrier, for example water for injection, immediately prior to use. Extemporaneous injection solutions and suspensions maybe prepared from sterile powders, granules and tablets of the type described above.

The pharmaceutical compositions of the present invention may also be used in the form of veterinary formulations, including those adapted for the following: (1) oral administration, for example, drenches (aqueous or nonaqueous solutions or suspensions), tablets, boluses, powders, granules or pellets for admixture with feed stuffs, pastes for application to the tongue; (2) parenteral administration, for example, by subcutaneous, intramuscular or intravenous injection as, for example, a sterile solution or suspension or, when appropriate, by intramammary injection where a suspension or solution is introduced into the udder of the animal via its teat; (3) topical application, for example, as a cream, ointment or spray applied to the skin; or (4) intravaginally, for example, as a pessary, cream or foam.

As mentioned above, certain embodiments of the present invention are directed to introduction of the ATMO group or moieties thereof to convert classes of β-lactams from substrates to inhibitors. While it was previously thought that such side chains will only confer inhibition to β-lactams (i.e., cephalosporins) that also have a displaceable R₂ side chain, this invention introduces the ATMO and other sterically-demanding substituents or groups onto, for example, the penicillin and carbacephem scaffolds, neither of which have a displaceable R₂ side chain. As shown herein, such new β-lactams, e.g., ATMO-penicillin and ATMO-loracarbef (FIG. 1B) were effective inhibitors, with IC₅₀ values of 900 nM and 80 nM respectively.

To investigate the structural basis for this inhibition, the structure of the ATMO-penicillin/AmpC complex was determined by X-ray crystallography to 1.72 Å resolution and compared to the structure of AmpC in complex with a substrate, amoxicillin, which was determined to 1.87 Å resolution. To investigate the biological relevance of these awkward inhibitors, the efficacy of cefotaxime and ATMO-loracarbef were also investigated in bacterial cell culture. These studies, as described more fully below, show a structural basis for the actions of these and other “awkward” inhibitors of serine β-lactamases, for use in the design of new agents to overcome bacterial resistance.

As mentioned above, an awkward inhibitor of this invention can be described as one that is recognized well-enough by an enzyme to form a covalent adduct, but does not quite fit in, and so finds it difficult to move on to the next stage of the reaction—in the context of the present invention, hydrolytic attack in the case of a β-lactam bound to a β-lactamase. Without limitation to any one theory, mechanism or mode of operation, if steric or structural awkwardness is a useful principal, it should be possible to use groups or substituents that confer such awkwardness with various other β-lactam structures to prepare new compositions, thereby converting substrates into inhibitors. The ability to convert two different classes of substrates for AmpC, a penicillin and a carbacephem, as demonstrated herein, into sub-micromolar inhibitors by addition of a bulky R₁ group, is consistent therewith. This and other aspects of the present invention may be understood by considering the kinetic and structural attributes of these inhibitors.

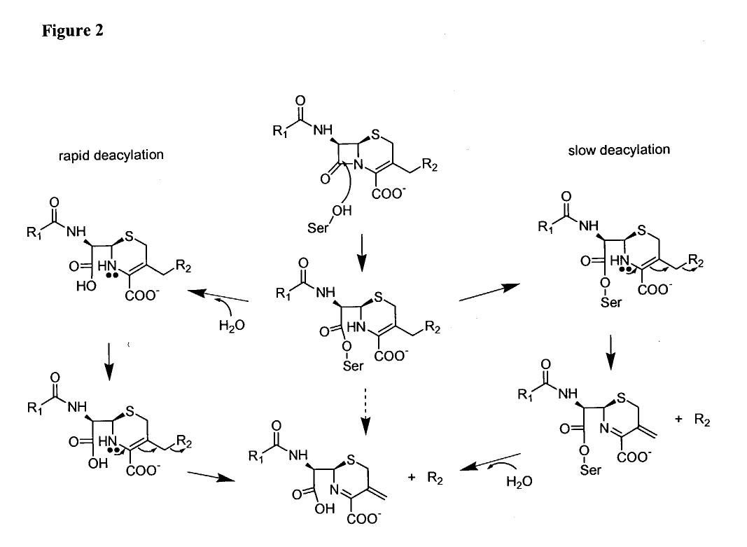

The hydrolytic rate constant, k_{cat}, for cefotaxime, ATMO-penicillin, and ATMO-loracarbef are low enough—10,000- to 100,000-fold reduced compared to their analogous substrates—that they may be usefully considered to be inhibitors of AmpC (Table 1). When tested in the presence of a good substrate such as cephalothin or nitrocefin, these ATMO-containing β-lactams have IC₅₀ values ranging from 900 nM for ATMO-penicillin to 80 nM for ATMO-loracarbef (Table 1). As mentioned above, the inhibition conferred by the ATMO group in 3^{rd} generation cephalosporins has been shown to be coupled to the ability of the R₂ side chain of these β-lactams to depart when in the acyl-adduct complex, rendering the resulting adduct less susceptible to hydrolytic attack (FIG. 2). Others have posited that it is not the leavability of this R₂ side chain but rather the electron-withdrawing inductive effect that this group has on the β-lactam core that is more important. Regardless, for representative ATMO-penicillin and ATMO-loracarbef compositions of this invention, which do not have a displaceable R₂ group, neither of these explanations holds—contrary to the prior art, the entire inhibitory effect may be attributed solely to the addition of a sterically-bulky side chain or substituent. This may be understood quantitatively by comparing the deacylation rate constants, k_{cat}, for the ATMO-bearing inhibitors to their analogous substrates that lack this group. The ratios of the turnover rates (Table 1) for each pair of β-lactams (ATMO-bearing inhibitor divided by non-ATMO-bearing substrate) can be compared:

$$\begin{matrix}\frac{cefotaxime {k}_{cat}}{cephalothin {k}_{cat}} & ( I ) \\ \frac{ATMO - penicillin {k}_{cat}}{penicillin G {k}_{cat}} & ( II ) \\ \frac{ATMO - loracarbef {k}_{cat}}{loracarbef {k}_{cat}} & ( III )\end{matrix}$$

Were the ratio (I) in the cephalosporin pair (with a readily displaceable acetate R₂ group) lower than that for the penicillin (II) or carbacephem (III) pairs (which have no displaceable R₂ group), then the rapid departure of the cephalosporin R₂ group would be key to trapping the complex in its acyl-enzyme state (since cefotaxime and cephalothin have identical R₂ groups), and the inhibitory effect of the ATMO group would not be fully transferable to β-lactams that lack an R₂ group. However, this is not observed: the k_{cat} ratio is actually larger for the cephalosporin pair (1.71×10⁻⁴) than for the penicillin pair (3.38×10⁻⁵) and very similar to the carbacephem pair (2.73×10⁻⁴), indicating that the ATMO-containing analogs for these β-lactams are just as or more stable to hydrolysis compared to their analogous substrate than is the cephalosporin (Table 1). Qualitatively, the same pattern may be seen if the IC₅₀ values for cefotaxime, ATMO-penicillin, and ATMO-loracarbef are compared—the value for ATMO-loracarbef is 11-fold lower than that of cefotaxime and the value for ATMO-penicillin is comparable to cefotaxime (IC₅₀ values convolute acylation and deacylation rate constants, and must be interpreted carefully).

Accordingly, the present invention can also provide a method of using a β-lactam core substituent to inhibit β-lactamase activity. Such a method comprises (1) providing compound comprising a β-lactam core structure and further comprising a substituent at the 6(7)-β-position of the core structure; and (2) contacting such a compound with a β-lactamase enzyme, the substituent having a steric effect sufficient to reduce the rate of deacylation of the β-lactam core structure complexed with such a β-lactamase. As discussed above as a distinction over the prior art, such a core structure can be absent a displaceable substituent at the C-3 position thereof. Without limitation, preferred embodiments include those compounds having penicillin and carbacephem core structures as can be substituted at the 6(7)-β-position thereof. The identity and structure of any such substituent is limited only by way of its complexation with a β-lactamase, the effect thereof on the rate of deacylation and/or resulting lactamase inhibition.

The results summarized above demonstrate the use and transferability of bulky R₁ groups that heretofore had been used only in the unrelated context of cephalosporins and monobactams (in the case of aztreonam). Accordingly, the new β-lactams of this invention can be extended to include any compound with the β-lactam core, substituted as illustrated, such compounds/compositions including but not limited to penicillins and carbacephems of the type demonstrated herein.

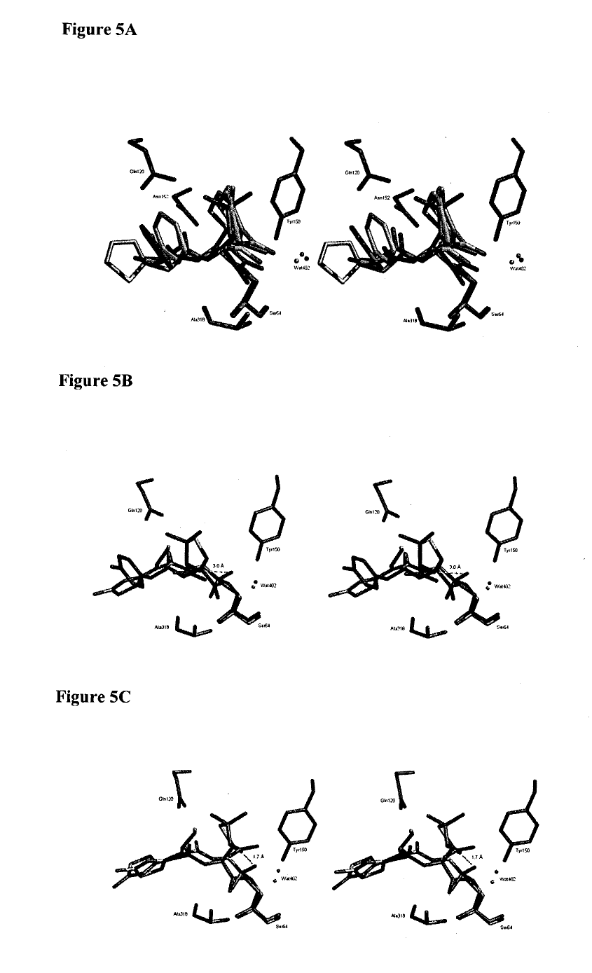

To provide a possible structural bases for how the ATMO group confers inhibition to the penicillins, for example, the structure of a penicillin substrate of AmpC was first determined. The complex between wild-type AmpC and amoxicillin is the first structure of a penicillin substrate in complex with a class C β-lactamase. Penicillin substrates bind in the active site of AmpC in a nearly identical manner as the carbacephem substrate loracarbef and the cephalosporin substrate cephalothin (FIG. 5A). In this, the class C β-lactamases appear different from the class A β-lactamases, where cephalosporins and penicillins are seen to adopt different configurations in the active site. The interactions between the R₁ amide group and conserved active site residues Gln120, Asn152, and Ala318 are retained, as are the positions of the β-lactam carbonyl oxygen. The 5-membered penicillin ring overlays closely with the 6-membered loracarbef and cephalothin rings, as do the ubiquitous carboxylate substituents on each of these rings. The putative deacylating water, Wat402, occupies a nearly identical location as that seen in the loracarbef and cephalothin complexes, implying a catalytic mechanism identical to that seen with the cephalosporins. Overlaying the structure of the amoxicillin/AmpC complex with that of a deacylation transition-state analog, ceftazidime boronic acid (FIG. 1), in complex with AmpC shows that the β-lactam ring nitrogen is positioned to stabilize the deacylation transition state complex, being 3.0 Å from the expected position of the deacylating water in the high energy intermediate (FIG. 5B). In short, the catalytically competent conformation of amoxicillin closely resembles that of carbacephem and cephalosporin substrates, and that the hydrolytic mechanism seems to be shared among these different classes of β-lactams.

The acyl-adduct structure of AmpC covalently bound to ATMO-penicillin closely resembles that when bound to the 3^{rd} generation cephalosporin ceftazidime (FIG. 1A). Like ceftazidime, the bulky ATMO group on the penicillin derivative appears to force the thiazolidine ring (the analog of the dihydrothiazine ring on the cephalosporin) into a conformation where it would block the formation of the high-energy deacylation intermediate. Although the ATMO R₁ group is somewhat smaller than that of ceftazidime, a comparison of the binding modes of ATMO-penicillin and ceftazidime indicates that the R₁ groups bind very similarly, with nearly identical positions for the amide groups, aminothiazole rings, and methoxime substituents. The additional bulk of the 1,1-dimethyl-1-carboxylate group of ceftazidime appears to only further displace the six-membered cephalosporin ring relative to the five-membered penicillin ring of ATMO-penicillin. The mechanism of inhibition observed is the same as was observed in the art with cloxacillin, ceftazidime, and moxalactam, namely the destabilization of the deacylation transition state. In contrast to the 3.0 Å distance between the β-lactam ring nitrogen of amoxicillin and the position of the deacylating water in the high-energy intermediate state, the analogous distance in the ATMO-penicillin complex would be a mere 1.7 Å (FIG. 5C). These atoms would be so close in space as to be in van der Waals violation; thus the formation of the deacylation transition state would be destabilized by the position of the ring nitrogen rather than stabilized, as it appears to be by the substrates.

Two conformers were observed in both the amoxicillin and ATMO-penicillin complexes with AmpC. FTIR spectroscopic studies have suggested that, in their acyl-adducts with β-lactamases, the carbonyl group that forms the ester with the catalytic serine (Ser64 in AmpC) can adopt more than one conformation. Until recently, there had been no crystallographic evidence for such libration within the acyl adduct. Recent X-ray structures involving both class A and class C β-lactamases have suggested that such alternate conformations can indeed be observed. The resolution of the structures reported here are sufficient to discern both the canonical conformation, with the carbonyl oxygen in the “oxyanion” or “electrophilic” hole (modeled at 75% occupancy in each structure) and the conformation with the carbonyl oxygen flipped out of the hole (modeled at 25% occupancy). The occurrence of these alternate conformations may partly explain why it has been possible to capture the acyl-adduct of the substrate amoxicillin in the wild-type crystal, as the activity of the enzyme clearly is slowed in the crystal environment.

From a drug/compositional/pharmaceutical design perspective, perhaps the most widely-applicable therapeutic result to emerge from the high-resolution structure of ATMO-penicillin in complex with AmpC is insight into the transferability of bulky R₁ groups, substituents or moieties thereof, to induce inhibition.—Examination of the binding modes of several ligands with bulky R₁ substituents shows surprisingly nearly identical modes of binding—and consequently inhibition—among various unrelated β-lactam families: monobactams, cephalosporins, and now penicillins. The size of the second ring—whether six-membered in the case of the cephalosporins, five-membered in the case of penicillins, or zero-membered in the case of monobactams—does not seem to affect inhibition. Accordingly, the observations made in association with this invention can be used in the design and synthesis of new awkward inhibitors of β-lactamases, and perhaps other classes of enzymes that go through a covalent adduct as part of their reaction mechanism. Indeed, in cell culture studies the MIC values of cefotaxime and ATMO-loracarbef were 1 μg/mL or less, even for bacteria that express β-lactamases—well within the therapeutic range.

### EXAMPLES OF THE INVENTION

The following non-limiting examples and data illustrate various aspects and features relating to the β-lactam side chains/substituents, compositions and/or methods of the present invention, including the substitution or modification of β-lactam molecular structures, such modifications as are available through the synthetic methodology as described herein. In comparison with the prior art, the present compositions and methods provide results and data which are surprising, unexpected and contrary thereto. While the utility of this invention is illustrated through the use of several preferred β-lactam molecular structures and substituents thereon, it will be understood by those skilled in the art that comparable results are obtainable with various other substituents, core structures and resulting compositions, as are commensurate with the scope of this invention.

Chemical Synthesis. All reactions were carried out in oven-dried glassware under an atmosphere of dry nitrogen.

Accession Numbers. Coordinates for amoxicillin and ATMO-penicillin in complex with AmpC have been deposited in the Protein Data Bank with corresponding accession codes.

### Example 1

ATMO-Penicillin. (Z)-(2-aminothiazol-4-yl)methoxyiminoacetic acid (1.01 g, 5 mMol) was slurried in DMF (15 mL) at room temperature. 2-Chloro-4,6-dimethoxy-1,3,5-triazine (966 mg, 5.5 mMol) and N-methylmorpholine (0.583 mL, 5.3 mMol) were added. The mixture was stirred at room temperature for 30 minutes at which time the system was a homogeneous solution of active ester. In a separate flask, the tosylate salt of allyl penicillanate(26) (2.31 g, 5.4 mMol) was slurried in CH₃CN (11 mL). N-methylmorpholine (1.21 mL, 11 mMol) was added and the mixture was stirred until homogeneous. The penicillin solution was added to the ATMO-active ester solution via syringe over approximately 2 minutes and the resulting acylation mixture was stirred at room temperature for 12 hours. The reaction mixture was diluted with EtOAc. The organic phase was washed with pH 4 buffer (3×) and brine (1×), dried MgSO₄, and concentrated to an oil.

The crude ATMO-penicillin allyl ester (2.23 g) was adsorbed on silica gel-60 (10 g) and chromatographed over silica gel-60 (10 g) using a gradient elution of CHCl₃ to 10% CH₃OH in CHCl₃. Appropriate fractions were combined and evaporated to yield an oil (1.83 g, 83%) with spectral characteristics (electrospray ionization mass spectrometry (ES/MS), ¹H NMR) consistent with the desired product, ATMO-penicillin allyl ester.

To a stirred solution of Pd(OAc)₂ (25 mg, 0.11 mMol) in CH₃CN (2 mL) was added a solution of PPh₃ (140 mg, 0.53 mMol) in CH₃CN (1.3 mL). A thick yellow-green precipitate formed in approximately 15 minutes. To the vigorously stirred precipitate was added (n-Bu)₃SnH (66 μL, 0.25 mMol). Stirring was continued for 30 minutes at which point a solution of ATMO-penicillin allyl ester (1.83 g, 4.16 mMol) in EtOAc (15 mL) was added in one portion. The reaction mixture immediately became homogeneous. A solution of sodium 2-ethylhexanoate in EtOAc (0.5 M, 10 mL) was added dropwise over approximately 5 minutes during which time a precipitate formed. The crude precipitated ATMO-penicillin sodium salt was isolated by low speed centrifugation of the reaction mixture. The solid was wash with ether (3×) and dried in vacuo to afford the crude salt (1.05 g).

Pure ATMO-penicillin was isolated by preparative HPLC and lyophilization of relevant fractions. Method: 19 mm×300 mm Waters Xterra C18 column (5 μm), 20 mL/min, gradient elution 5-40% CH₃OH in aq NH₄HCO₃ over 30 minutes. ES/MS (positive ion) 400.1 [M+H], 240.9 [β-lactam “vertical cleavage”] (negative ion) 398.1 [M−H]; ¹H NMR at 400 MHz in DMSO-d₆ (ppm δ, multiplicity/integration, J Hz): 1.39, s 3H; 1.49, s 3H, 3.74, s 3H, 5.39, d 1H, J=3.9; 5.43, d/d 1H, J=3.9/7.4; 6.68, s 1H; 9.36, d 1H, J=7.4.

### Example 2

ATMO-loracarbef. 7-ATMO-3-chloro-carbacephem was prepared in an analogous manner with the analytical sample isolated by preparative HPLC. ES/MS (positive ion) 385.2 [M+H], 406.9 [M+Na]; (negative ion) 383.1 [M−H]; ¹H NMR at 400 MHz in DMSO-d₆ (ppm δ, multiplicity/integration, J Hz): 1.81, m 2H; 2.70, m 2H; 3.69, s 3H; 3.78, m 1H, 5.11, d/d 1H, J=5.0/7.3; 6.65, S 1H; 9.27, d 1H, J=7.3.

### Example 3a

Analogous ATMO-substituted penicillin compounds can be prepared with choice of the corresponding iminoacetic acid or alkoxyiminoacetic acid, using straight-forward modifications of the synthetic procedure provided above. Likewise, the allyl-derivatives of various other β-lactam core structures can be used with available iminoacetic acid reagents to provide a range of analogous ATMO-substituted β-lactam inhibitor compounds, in accordance with this invention.

### Example 3b

Similarly, a range of penicillin and carbacephem related compounds, in accordance with this invention and the foregoing steric effects, can be prepared from amoxicillin and loracarbef, respectively, via alkylation or acylation with a corresponding agent selected to provide a desired steric/substituent effect, as would be understood by those skilled in the art made aware of this invention, using known reagents, starting materials and synthetic techniques. For example and without limitation, compounds 3 and 6 (FIG. 1B) can be prepared from amoxicillin or loracarbef, respectively (each of which is either commercially-available or prepared as provided in the literature), by reaction with the corresponding dione. Various other amino-substituted amoxicillin or loracarbef analogs are contemplated, in accordance with this invention, such compounds limited only by steric effect consistent with the preceding discussion and resulting lactamase inhibition.

### Example 4

Escherichia coli. AmpC β-lactamase was expressed and purified to homogeneity as previously described. The AmpC-catalyzed hydrolysis of the various β-lactams was monitored in an HP8453 UV/visible spectrophotometer. For cephalothin (Sigma, St. Louis, Mo.) and cefotaxime (Sigma), reactions were monitored at 265 nm, penicillin G (Sigma) and ATMO-penicillin at 235 nm, loracarbef at 262 nm, and ATMO-loracarbef at 260 nm. The reaction buffer used during the assays was 50 mM tris hydroxymethyl aminomethane hydrochloride (Fisher, Fair Lawn, N.J.) at pH 7 in doubly-deionized water. k_{cat} and K_{M} values were determined at an enzyme concentration of 1.75 nM, with enzyme concentration determined, from more concentrated stocks, based on an ε₂₈₀ of 0.098 μM⁻¹cm⁻¹. Where K_{M} values could not be determined because they were so low (i.e., for molecules that behaved more like inhibitors), IC₅₀ values were determined against 200 μM cephalothin for ATMO-penicillin and cefotaxime, and 200 μM nitrocefin (Oxoid, Ogdensburg, N.Y.) in the case of ATMO-loracarbef. Except for compounds synthesized for this study (ATMO-penicillin, ATMO-loracarbef), all compounds were used as supplied from the manufacturers without further purification.

### Example 5

Enzymology. The AmpC-catalyzed hydrolysis of the β-lactams cefotaxime, ATMO-penicillin, and ATMO-loracarbef (FIG. 1B) were monitored by UV-vis spectroscopy (Table 1). Consistent with the “awkwardness” hypothesis, all of these ATMO-bearing β-lactams were good inhibitors for AmpC. Indeed, the k_{cat} of ATMO-penicillin was so low that we were are only able to assign an upper bound of 0.0045 s⁻¹ for this value. Further, the K_{M} was too low to be determined for these ATMO-bearing inhibitors, and instead we use the inhibition IC₅₀ values for these compounds when reporting the turnover rate.

To isolate the influence of the bulky R₁ groups on the strong inhibition of these compounds, the turnover rates of β-lactam substrates from the same β-lactam families were determined as well. Cephalothin (for comparison to cefotaxime), penicillin G (for comparison to ATMO-penicillin), and loracarbef (for comparison to ATMO-loracarbef) were good substrates for AmpC, with k_{cat} values of 263 s⁻¹, 133 s⁻¹, and 118 s⁻¹, respectively, about 10⁴-10⁵-fold faster than their ATMO-bearing analogs (Table 1).

### Example 6

Crystal Growth and Structure Determination. Purified AmpC was crystallized in 1.7 M potassium phosphate (KPᵢ) buffer at pH 8.7 as described. AmpC crystals were harvested and placed in a 6 μL drop of 1.7 M KPᵢ (pH 8.7) and soaked with excess ligand (50-70 mM) for 15 minutes and then transferred to a fresh drop for another 15 minutes to obtain the acyl-enzyme complexes presented here. The crystals were then transferred to a pH 8.7 cryoprotectant solution containing 20% sucrose, 1.7 M KPᵢ, and excess ligand (50 mM) for 10-15 seconds before being flash-frozen in liquid nitrogen.

Diffraction data were collected at DND-CAT beamline 5-IDB at the Advanced Photon Source at 100 K using a Mar-CCD detector. Reflections were indexed, integrated, and scaled using HKL software (Table 2). The complexes crystallized in the C2 space group, with two AmpC molecules per asymmetric unit, each containing 358 amino acid residues. 715 out of a possible 716 residues were included in each final model. The initial model was built by molecular substitution using an apo-AmpC structure (Protein Data Bank accession code 1KE4) without solvent molecules. The starting model was refined with the CNS software package, using rigid body, simulated annealing, positional minimization, and individual B-factor refinement. The maximum likelihood target was used during refinement, including a bulk solvent correction and a 2σ cutoff for data. Manual model building into sigma-A weighted electron density maps using O was alternated with rounds of positional and B-factor refinement in CNS.

Molecule two of the asymmetric unit exhibited stronger electron density for the ligands in both structures. Ligands were built into the 2|F_{O}|-|F_{C}| and |F_{O}|-|F_{C}| difference density in molecule two of both structures. Simulated-annealing omit density was also used to guide placement of the ligands in the active site.

### Example 7

Crystal structure of AmpC in complex with amoxicillin. The crystal structure of amoxicillin (FIG. 1) covalently bound to AmpC was determined to a resolution of 1.87 Å (Table 2). 91.3% of the amino acid residues were in the most favored regions of the Ramachandran plot, and the remaining 8.7% were in additionally allowed regions, excluding glycine and proline residues. The final R_{cryst} and R_{free} values of the refined model were 19.9% and 22.3%, respectively.

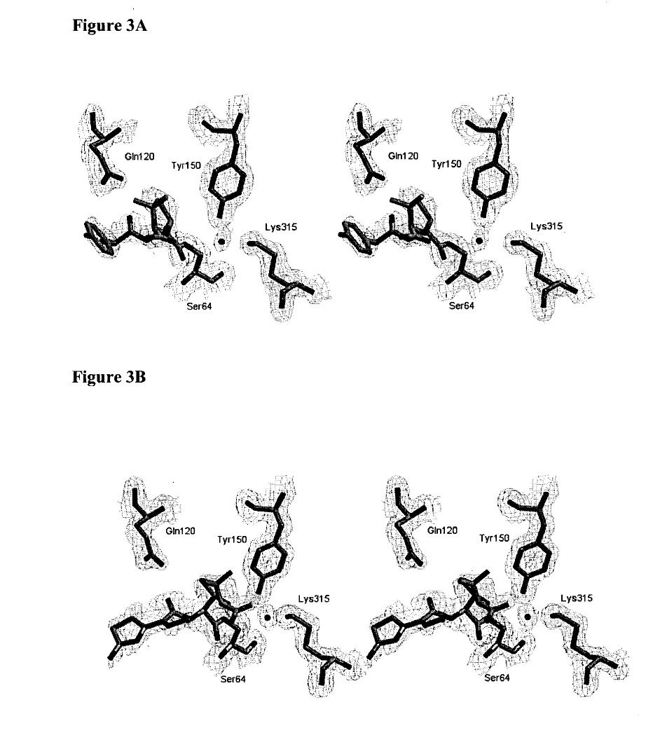

Due to the high resolution of this structure, we were able to distinguish two distinct conformations of amoxicillin in its covalent complex. The predominant confirmation (shown in FIG. 3a, modeled at 75% occupancy) shows the β-lactam carbonyl oxygen oriented in a catalytically competent confirmation in the “oxyanion” or “electrophilic” hole formed by the backbone amide groups of Ser64 and Ala318. The other conformation shows this oxygen swung “out” of the hole in a catalytically incompetent conformation; the rest of the ligand position remains mostly unperturbed in the active site. The transient existence of multiple conformations of the acyl-enzyme species has been suggested previously by both prior FTIR and crystallographic studies. Further discussion will focus on the catalytically competent conformation of the ligand.

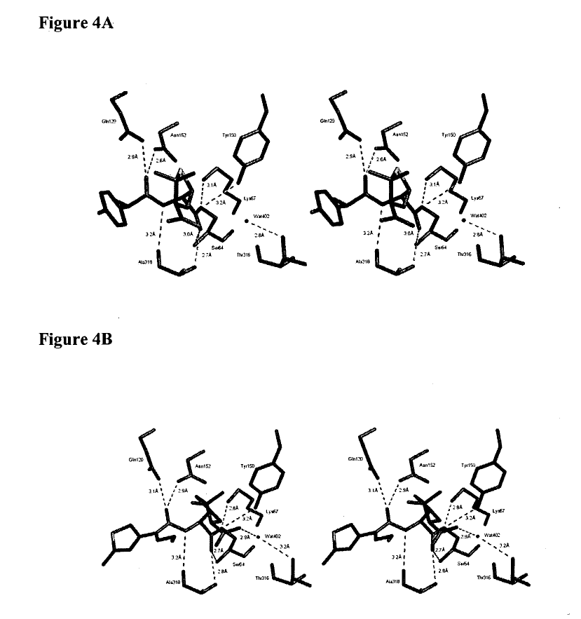

Key hydrogen-bonding interactions in the active site (FIG. 4A) closely resemble those typically seen in covalent complexes of β-lactams with AmpC. These include the key interactions between the amide group of the R₁ side chain (FIG. 1) and the conserved residues Gln120, Asn152, and Ala318. The putative deacylating water, Wat402, is also clearly observed and is stabilized by its interaction with Thr316. The position of Wat403, also important in the catalytic mechanism of AmpC, is also nearly identical to those seen in other substrate complexes.

### Example 8

Crystal structure of AmpC in complex with ATMO-penicillin. The crystal structure of ATMO-penicillin (FIG. 1) covalently bound to AmpC was determined to a resolution of 1.72 Å (Table 2). Excluding glycine and proline residues, 92.8% of the amino acid residues were in the most favored regions of the Ramachandran plot and 7.2% were in additionally allowed regions. The final R_{cryst} and R_{free} values of the refined model were 17.8% and 19.9%, respectively. As with the amoxicillin complex, two distinct conformations of the acyl-enzyme species were captured in the crystal structure: a more predominant conformation with the β-lactam carbonyl oxygen in the electrophilic hole (FIG. 3B, 75% occupancy), and a less common (25% occupancy) conformation with the β-lactam carbonyl oxygen swung out of the electrophilic hole.

Key hydrogen-bonding interactions in the active site (FIG. 4b) in this complex resembled other covalent complexes of β-lactams with AmpC. A difference between ATMO-penicillin and substrate complexes, such as those with loracarbef, cephalothin, and amoxicillin (above) is that the entire inhibitor is rotated such that the C3 carboxylate and the thiazolidine ring nitrogen occupy positions different from those adopted by substrate β-lactams. This conformation resembles that adopted by ceftazidime in its complex with AmpC, and like ceftazidime seems to owe to interactions between the ATMO group and highly conserved residues at the distal end of the AmpC site, such as Val211 and Tyr221. The putative deacylating water is still observed in this complex, stabilized by both Thr316 and a β-lactam carboxylate oxygen.

### Example 9

Microbiology. Serial dilution assays in ligand culture were performed to examine the efficacy of these awkward β-lactams against clinically relevant pathogens grown in liquid culture (Table 3). The compounds were dissolved in KPᵢ or tris hydroxymethyl aminomethane hydrochloride to a concentration of 20-50 mM, and serial dilutions were performed into Luria Broth (Difco, Detroit, Mich.). Each broth solution was then inoculated with bacterial cells from an overnight culture that had been diluted to give an inoculum concentration of approximately 5×10⁵ CFU/mL. Against β-lactamase-expressing E. coli, cefotaxime had an MIC of 1/32 μg/mL and ATMO-loracarbef had an MIC of 1 μg/mL, both much improved compared to the analagous β-lactams cephalothin and loracarbef, which had MIC values of 64 and 8 μg/mL, respectively. As expected, the ATMO-bearing compounds also showed good efficacy against β-lactamase-negative E. coli, with MIC values of 1/128 μg/mL and ¼ μg/mL, respectively. The MIC of the test compounds was also determined against both β-lactamase-expressing and β-lactamase-negative strains of JM109 E. coli, and β-lactamase-expressing clinical isolates of E. cloacae and S. aureus, with results comparable to those provided herein.

TABLE 1
Kinetic data for hydrolysis of
analogous β-lactams by AmpC β-lactamase

| | ATMO | | |
|---|---|---|---|
| Compound | Containing? | K_{cat} (s⁻¹) | K_{M} (μM) |
| Cephalothin | No | 262.5 | 31.3 |
| Cefotaxime | Yes | 0.0448 | 0.80^{a} |
| Penicillin G | No | 133.1 | 5.05 |
| ATMO-penicillin | Yes | <0.0045 | 0.90^{a} |
| Loracarbef | No | 118.3 | 23.7 |
| ATMO-loracarbef | Yes | 0.0323 | 0.080^{a} |

^{a}IC₅₀ reported instead of K_{M}.

TABLE 3
Minimum inhibitory concentrations of various
β-1actams against clinically
relevant strains of bacteria.

| | | | | ATMO- |
|---|---|---|---|---|
| | Cephalothin | Cefotaxime | Loracarbef | Loracarbef |
| E. coli | 64 | 1/32 | 8 | 1 |
| expressing | | | | |
| AmpC | | | | |
| β-lactamase | | | | |
| E. coli | 8 | 1/128 | 4 | ¼ |
| not | | | | |
| expressing | | | | |
| AmpC | | | | |
| β-lactamase | | | | |

TABLE 2
Data collection and refinement statistics

| | AmpC + amoxicillin | AmpC + ATMO-penicillin |
|---|---|---|
| Space group | C2 | C2 |
| Unit cell | a = 118.33, b = 76.85 | a = 118.66, b = 76.73 |
| dimensions | | |
| (Å, deg) | | |
| | c = 97.99, β = 116.51 | c = 98.17, β= 116.19 |
| Number of | 2 | 2 |
| complexes per | | |
| asymmetric unit | | |
| Resolution (Å) | 1.87 | 1.72 |
| Number of observed | 225850 | 270758 |
| reflections | | |
| Number of unique | 63012 | 81383 |
| reflections | | |
| Completeness (%)^{a} | 97.0 (97.0) | 97.0 (95.3) |
| R_{merge} (%)^{a} | 5.6 (37.3) | 3.5 (24.3) |
| <I/σ_{I}>^{a} | 30.70 (3.92) | 31.49 (4.36) |
| Number of working | 56774 | 74290 |
| reflections | | |
| Resolution | 20.0-1.87 (1.91-1.87) | 20.0-1.72 (1.76-1.72) |
| range for | | |
| refinement (Å)^{a} | | |
| Number of | 715 | 715 |
| protein residues | | |
| Number of | 353 | 598 |
| water molecules | | |
| Rmsd for bond | 0.0057 | 0.0126 |
| lengths (Å) | | |
| Rmsd for bond | 1.34 | 1.71 |
| angles (deg) | | |
| R_{cryst} (%) | 19.9 | 17.8 |
| R_{free} (%)^{b} | 22.3^{b} | 19.9^{c} |
| Average | | |
| B-factor (Å²) | | |
| Protein | 29.16 | 23.62 |
| Ligand | 35.70 | 29.46 |
| Solvent | 29.60 | 29.94 |

^{a}Values in parentheses are for the highest-resolution shell used in refinement.
^{b}R_{free} was calculated with 2048 reflections set aside randomly.
^{c}R_{free} was calculated with 2324 reflections set aside randomly.

We claim:

1. A β-lactamase inhibitor comprising a compound selected from compounds of the formula 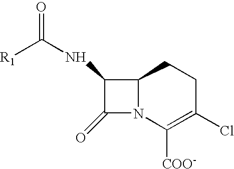
and compounds of the formula 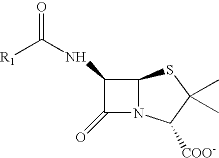
wherein R₁ is selected from aminothiazole oxime substituents and N-substituted benzylamine substituents.

2. The β-lactamase inhibitor of claim 1 wherein R₁ is selected from substituents of the formula 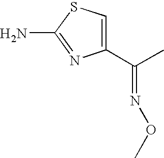
and substituents of the formula

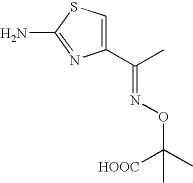

3. The β-lactamase inhibitor of claim 1 wherein R₁ is selected from substituents of the formula 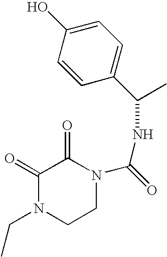
and substituents of the formula

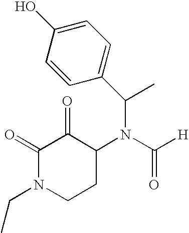

4. A β-lactamase inhibitor compound of the formula 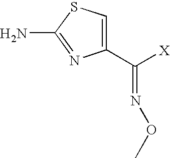
wherein R₂ is a moiety selected from alkyl and substituted alkyl moieties and X comprises a β-lactam core structure, said lactam structure not a cephalosporin and not a monobactam.

5. The compound of claim 4 wherein X comprises a penicillin core structure.

6. The compound of claim 5 wherein R₂ is alkyl.

7. The compound of claim 4 wherein X comprises a carbacephem core structure.

8. The compound of claim 7 wherein R₂ is a carboxy-substituted alkyl.

9. A method of inhibiting a β-lactamase comprising contacting a β-lactamase with an effective amount of a compound selected from compounds of the formula 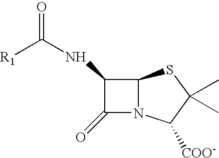
and compounds of the formula 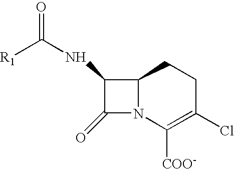
wherein R₁ is selected from aminothiazole oxime substituents and N-substituted benzylamine substituents.

10. The method of claim 9 wherein R₁ is selected from substituents of the formula 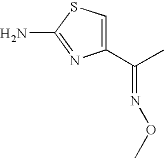
and substituents of the formula

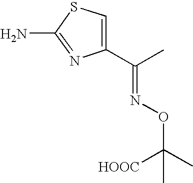

11. The method of claim 9 wherein R₁ is selected from substituents of the formula 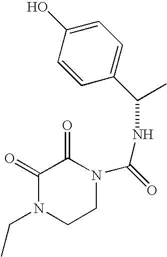
and substituents of the formula

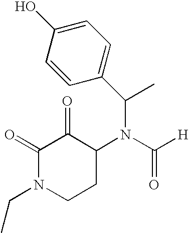

12. The method of claim 9 wherein the β-lactamase is produced by bacteria and said compound comprises a pharmaceutically-acceptable salt.

13. The method of claim 9 wherein said contact is in vivo.

14. A method of using a β-lactam core substituent to inhibit β-lactamase activity, said method comprising:
providing a compound with a β-lactam core structure comprising a substituent at the 6(7)-β-position of said core structure, said substituent having a steric effect sufficient to reduce the rate of deacylation of said β-lactam core structure complexed with a β-lactamase, said core structure absent a displaceable substituent at the C-3 position of said structure.

15. The method of claim 14 wherein said 6(7)-β-position substituent is selected from substituents of the formula 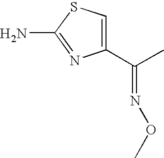
and substituents of the formula

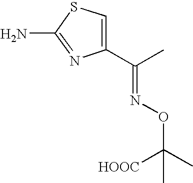

16. The method of claim 14 wherein said 6(7)-β-position substituent is selected from substituents of the formula 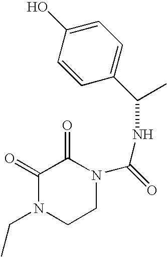
and substituents of the formula

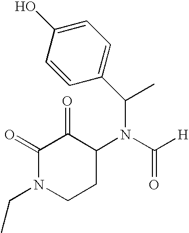

17. The method of claim 14 wherein said β-lactam core structure is of the formula

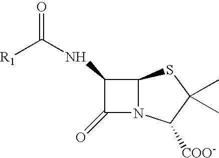

18. The method of claim 17 wherein R₁ is selected from

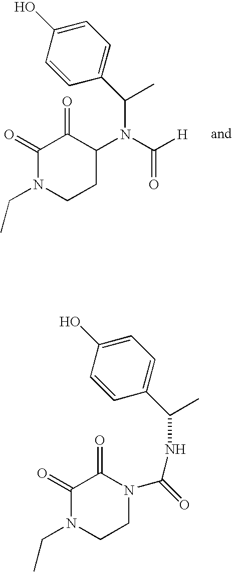

19. The method of claim 14 wherein said β-lactam core structure is of the formula

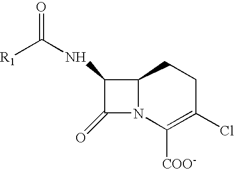

20. The method of claim 19 wherein R₁ is selected from

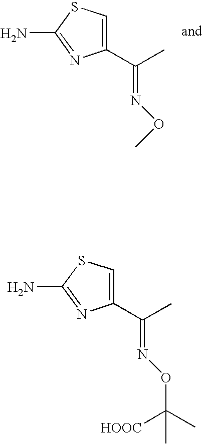

21. A pharmaceutical composition comprising a pharmaceutically-acceptable carrier and a compound selected from compounds of the formula 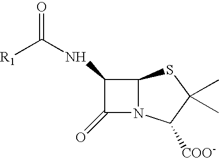
and compounds of the formula 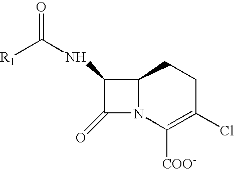
wherein R₁ is selected from aminothiazole oxime substituents and N-substituted benzylamine substituents.

22. The composition of claim 21 wherein R₁ is selected from substituents of the formula 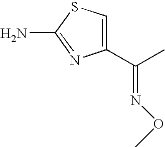
and substituents of the formula

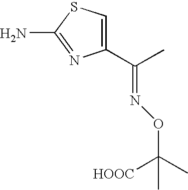

23. The composition of claim 21 wherein R₁ is selected from substituents of the formula 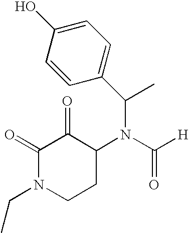
and substituents of the formula

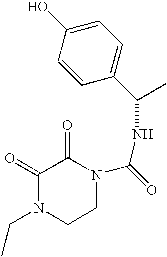

24. The composition of claim 21 further comprising a β-lactam antibiotic.
